(19) United States
(12) Patent Application Publication
(10) Pub. No.: US 2022/0407111 A1
(43) Pub. Date: Dec. 22, 2022

(54) ELECTROLYTE ADDITIVES FOR LITHIUM-ION BATTERIES

(71) Applicant: Apple Inc., Cupertino, CA (US)

(72) Inventors: Paul M. Bayley, Santa Clara, CA (US); Libo Hu, Mountain View, CA (US); Hongli Dai, Los Altos, CA (US); Zhengcheng Zhang, Naperville, IL (US); Qian Liu, Darien, IL (US); Khalil Amine, Oak Brook, IL (US); Mingfu He, Palos Park, IL (US)

(21) Appl. No.: 17/830,951

(22) Filed: Jun. 2, 2022

Related U.S. Application Data
(60) Provisional application No. 63/211,836, filed on Jun. 17, 2021.

(51) Int. Cl. H01M 10/0567; H01M 10/0525; H01M 10/0568; H01M 10/0569; C07D 207/14

(52) U.S. Cl. CPC H01M 10/0567; H01M 10/0525; H01M 10/0568; H01M 10/0569; C07D 207/14; H01M 2300/0042

(57) ABSTRACT

This disclosure relates generally to battery cells, and more particularly, electrolyte additives for use in lithium ion battery cells.

### CROSS-REFERENCE TO RELATED PATENT APPLICATION

This patent application claims the benefit under 35 U.S.C. § 119(e) of U.S. Patent Application No. 63/211,836, entitled “Electrolyte Additives for Lithium Ion Batteries”, filed on Jun. 17, 2021, which is incorporated herein by reference in its entirety.

### U.S. GOVERNMENT LICENSE RIGHTS

This invention was made with U.S. government support under WFO Proposal No. 85C85. This invention was made under a CRADA 1500801 between Apple Inc. and Argonne National Laboratory operated for the United States Department of Energy. The U.S. government has certain rights in the invention.

### FIELD

This disclosure relates generally to battery cells, and more particularly, electrolyte additives for use in lithium ion battery cells.

### BACKGROUND

Li-ion batteries are widely used as the power sources in consumer electronics. Consumer electronics need Li-ion batteries which can deliver higher volumetric energy densities and sustain more discharge-charge cycles. A Li-ion battery typically works at a voltage up to 4.45 V (full cell voltage).

A battery life cycle can deteriorate due to instability of cathode structure and electrolyte degradation. The cathode material stability can be improved by the modification of LiCoO₂ such as doping and surface coating. Limited progress has been made in developing electrolytes that can enable both high volumetric energy densities and long battery cycling life. Most existing electrolytes suffer from poor ability to form stable cathode-electrolyte (CEI) and/or solid-electrolyte interphases (SEI), leading to fast interfacial impedance growth and capacity decay.

### SUMMARY

In a first aspect, the disclosure is directed to an electrolyte fluid electrolyte fluid comprising a compound of Formula (I):

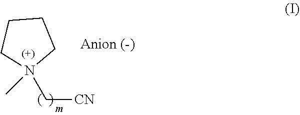

wherein m is an integer greater than or equal to 1 and less than or equal to 5. In various aspects, m can be greater than 2 and less than 4, equal to 3. The anion can be any anion known in the art, including those described herein.

In a second aspect, the compound is the compound of Formula (II)

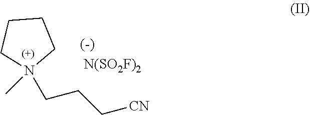

In a third aspect, the electrolyte fluid includes an electrolyte salt selected from LiPF₆, LiBF₄, LiClO₄, LiSO₃CF₃, LiN(SO₂F)₂, LiN(SO₂CF₃)₂, LiBC₄O₈, Li[PF₃(C₂CF₅)₃], LiC(SO₂CF₃)₃, and a combination thereof.

In a fourth aspect, the electrolyte fluid includes a solvent selected from ethylene carbonate (EC), dimethyl carbonate (DMC), diethyl carbonate (DEC), ethyl-methyl carbonate (EMC), ethyl propionate (EP), butyl butyrate (BB), methyl acetate (MA), methyl butyrate (MB), methyl propionate (MP), propylene carbonate (PC), ethyl acetate (EA), propyl propionate (PP), butyl propionate (BP), propyl acetate (PA), and butyl acetate (BA), and a combination thereof.

In a fifth aspect, the electrolyte fluid includes an additive selected from (LiDFOB), pro-1-ene-1, 3-sultone (PES), methylene methanedisulfonate (MMDS), vinyl ethylene carbonate (VEC), propane sultone (PS), fluoroethylene carbonate (FEC), succinonitrile (SN), vinyl carbonate (VC), adiponitrile (ADN), ethyleneglycol bis(2-cyanoethyl)ether (EGPN), 1,3,6-hexanetricarbonitrile (HTCN), and a combination thereof.

In a sixth aspect, the disclosure is directed to a battery cell. The battery cell can include a cathode having a cathode active material disposed on a cathode current collector, and an anode having an anode active material disposed on an anode current collector. The anode is oriented towards the cathode such that the anode active material faces the cathode active material. A separator is disposed between the cathode active material and the anode active material. An electrolyte fluid as described herein is disposed between the cathode and anode. In some variations, the battery cell can have a higher than the specific discharge capacity at 25° C. of a battery cell comprising the electrolyte fluid that does not include a compound of Formula (I). In additional variations, the energy retention of the battery cell at cycle 200 is at least 20% higher than the energy retention of a battery cell comprising the electrolyte fluid in the absence of the compound of Formula (I). In still further variations, the RSS of the battery cell at cycle 200 is at least 40% decreased than the compared a battery cell comprising the electrolyte fluid in the absence of the compound of Formula (I).

### BRIEF DESCRIPTION OF THE DRAWINGS

The disclosure will be readily understood by the following detailed description in conjunction with the accompanying drawings, wherein like reference numerals designate like structural elements, and in which:

FIG. 1 is a top-down view of a battery cell, in accordance with illustrative embodiments;

FIG. 2 is a perspective view of a battery cell, in accordance with illustrative embodiments;

FIG. 3 depicts specific discharge capacity for a battery containing a control electrolyte formulation compared to a battery containing the control electrolyte formulation with a 1% Pyr14CNFSI, in accordance with illustrative embodiments;

FIG. 4 depicts the cycling energy retention at cycle 200 for a battery operating at 45° C. of the control electrolyte as compared to the control including 1% Pyr14CNFSI, in accordance with illustrative embodiments;

FIG. 5 depicts the cycling RSS at cycle 202 at cycle 200 for a battery operating at 45° C. a battery having control electrolyte and the control electrolyte including 1% Pyr14CNFSI, in accordance with illustrative embodiments;

FIG. 6 is a plot of 0.2 C energy retention normalized to cycle 26 as a function of cycle count at 45° C. for a battery having a control electrolyte and an electrolyte including 1% Pyr14CNFSI, in accordance with illustrative embodiments; and

FIG. 7 depicts a plot of RSS as a function of battery cycle count at 45° C. for a battery having a control electrolyte and an electrolyte including 1% Pyr14CNFSI, in accordance with illustrative embodiments.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

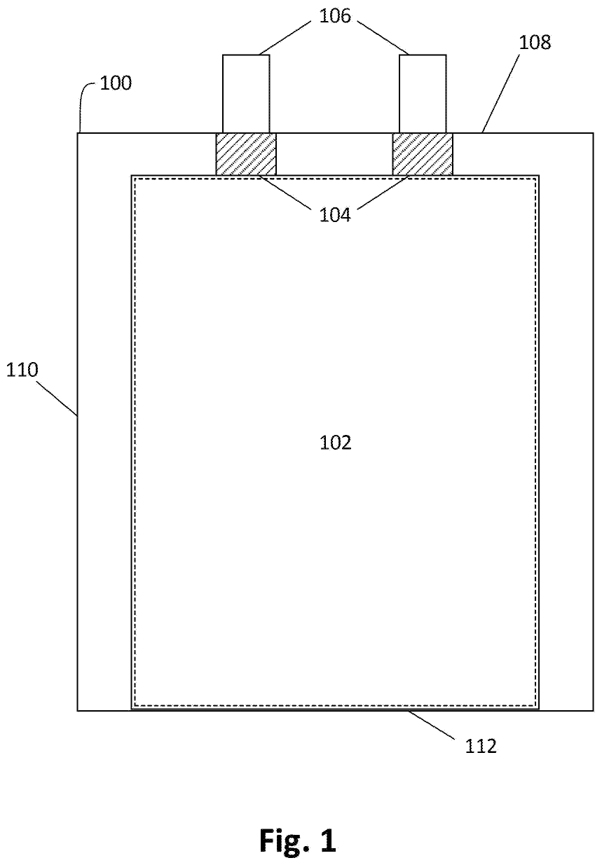

FIG. 1 presents a top-down view of a battery cell 100 in accordance with an embodiment. The battery cell 100 may correspond to a lithium-ion or lithium-polymer battery cell that is used to power a device used in a consumer, medical, aerospace, defense, and/or transportation application. The battery cell 100 includes a stack 102 containing a number of layers that include a cathode with a cathode active coating, a separator, and an anode with an anode active coating. More specifically, the stack 102 may include one strip of cathode active material (e.g., aluminum foil coated with a lithium compound) and one strip of anode active material (e.g., copper foil coated with carbon). The stack 102 also includes one strip of separator material (e.g., a microporous polymer membrane or non-woven fabric mat) disposed between the one strip of cathode active material and the one strip of anode active material. The cathode, anode, and separator layers may be left flat in a planar configuration or may be wrapped into a wound configuration (e.g., a “jelly roll”). An electrolyte solution is disposed between each cathode and anode.

During assembly of the battery cell 100, the stack 102 can be enclosed in a pouch or container. The stack 102 may be in a planar or wound configuration, although other configurations are possible. In some variations, the pouch such as a pouch formed by folding a flexible sheet along a fold line 112. In some instances, the flexible sheet is made of aluminum with a polymer film, such as polypropylene. After the flexible sheet is folded, the flexible sheet can be sealed, for example, by applying heat along a side seal 110 and along a terrace seal 108. The flexible pouch may be less than or equal to 120 microns thick to improve the packaging efficiency of the battery cell 100, the density of battery cell 100, or both.

The stack 102 can also include a set of conductive tabs 106 coupled to the cathode and the anode. The conductive tabs 106 may extend through seals in the pouch (for example, formed using sealing tape 104) to provide terminals for the battery cell 100. The conductive tabs 106 may then be used to electrically couple the battery cell 100 with one or more other battery cells to form a battery pack. For example, the battery pack may be formed by coupling the battery cells in a series, parallel, or a series-and-parallel configuration. Such coupled cells may be enclosed in a hard case to complete the battery pack, or may be embedded within an enclosure of a portable electronic device, such as a laptop computer, tablet computer, mobile phone, personal digital assistant (PDA), digital camera, and/or portable media player.

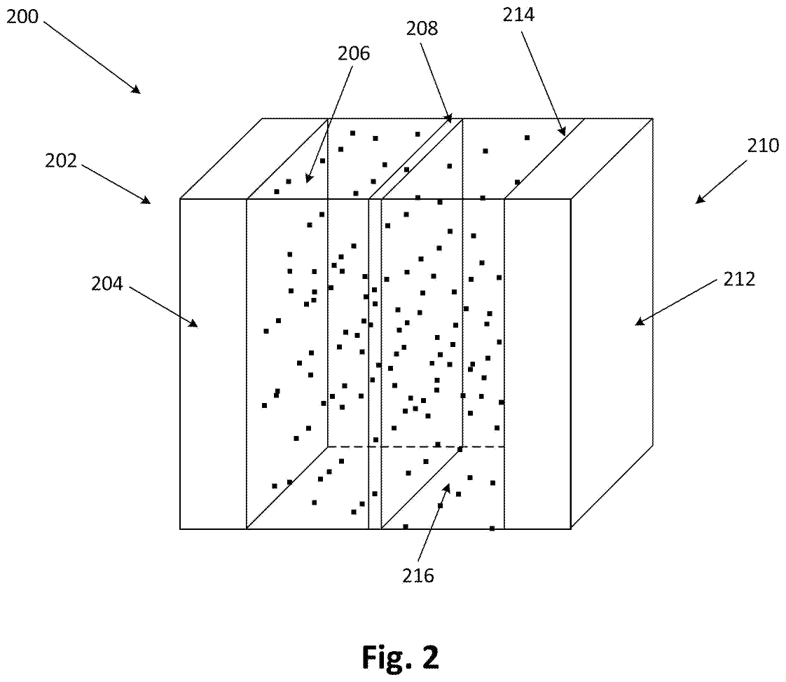

FIG. 2 presents a perspective view of battery cell 200 (e.g., the battery cell 100 of FIG. 1) in accordance with the disclosed embodiments. The battery includes a cathode 202 that includes current collector 204 and cathode active material 206 and anode 210 including anode current collector 212 and anode active material 214. Separator 208 is disposed between cathode 202 and anode 210. Electrolyte fluid 216 is disposed between cathode 202 and anode 210, and is in contact with separator 208. To create the battery cell, cathode 202, separator 208, and anode 210 may be stacked in a planar configuration, or stacked and then wrapped into a wound configuration. Electrolyte fluid 216 is then added. Before assembly of the battery cell, the set of layers may correspond to a cell stack.

The cathode current collector, cathode active material, anode current collector, anode active material, and separator may be any material known in the art. In some variations, the cathode current collector may be an aluminum foil, the anode current collector may be a copper foil. The cathode active material can be any material described in, for example, Ser. No. 14/206,654, 15/458,604, 15/458,612, 15/709,961, 15/710,540, 15/804,186, 16/531,883, 16/529,545, 16/999,307, 16/999,328, 16/999,265, each of which is incorporated herein by reference in its entirety.

The separator may include a microporous polymer membrane or non-woven fabric mat. Non-limiting examples of the microporous polymer membrane or non-woven fabric mat include microporous polymer membranes or non-woven fabric mats of polyethylene (PE), polypropylene (PP), polyamide (PA), polytetrafluoroethylene (PTFE), polyvinyl chloride (PVC), polyester, and polyvinylidene difluoride (PVdF). However, other microporous polymer membranes or non-woven fabric mats are possible (e.g., gel polymer electrolytes).

In general, separators represent structures in a battery, such as interposed layers, that prevent physical contact of cathodes and anodes while allowing ions to transport therebetween. Separators are formed of materials having pores that provide channels for ion transport, which may include absorbing an electrolyte fluid that contains the ions. Materials for separators may be selected according to chemical stability, porosity, pore size, permeability, wettability, mechanical strength, dimensional stability, softening temperature, and thermal shrinkage. These parameters can influence battery performance and safety during operation.

In general, electrolyte fluid can act a conductive pathway for the movement of cations passing from the negative to the positive electrodes during discharge. The electrolyte fluid includes an electrolyte salt, electrolyte solvent, and one or more electrolyte additives.

In some variations, the disclosure is directed to electrolyte fluids that include cyano-functionalized ionic liquid compounds. In various aspects, battery cells that incorporate these electrolyte fluids can have one or more properties, including larger initial capacities, longer cycle life, and reduced cell impedance growth, and/or increased recovered capacities after storage as compared to a battery cell containing an electrolytic fluid not including a cyano-functionalized ionic liquid compound.

In some variations, the ionic liquid compound is a compound of Formula (I):

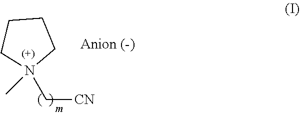

wherein m is an integer greater than or equal to 1 and less than or equal to 5.

In some variations, m is 1. In some variations, m is 2. In some variations, m is 3. In some variations, m is 4. In some variations, m is 5. In some variations, m is greater than or equal to 1 and less than or equal to 3.

The anion can be any anion used in ionic liquids. In some variations, the anion can be an inorganic anion (e.g., N(SO₂F)₂, N(SO₂CF₃)₂, Cl⁻, AlCl₄⁻, PF₆⁻, BF₄⁻, NTf₂⁻, OTF, N(CN)₂⁻, HSO₄⁻, DCA⁻) or and organic anion (e.g., CH₃COO⁻, CH₃CH₂OSO₃⁻, CH₃SO₃⁻).

In some more specific variations, the ionic liquid compound of Formula (I) is the compound of Formula (II):

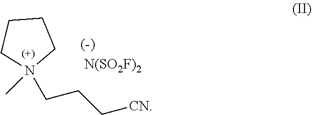

In some variations, the compound of Formula (I) is at least 0.1 wt % of the total electrolyte fluid. In some variations, the compound of Formula (I) is at least 0.2 wt % of the total electrolyte fluid. In some variations, the compound of Formula (I) is at least 0.4 wt % of the total electrolyte fluid. In some variations, the compound of Formula (I) is at least 0.6 wt % of the total electrolyte fluid. In some variations, the compound of Formula (I) is at least 0.8 wt % of the total electrolyte fluid. In some variations, the compound of Formula (I) is at least 1.0 wt % of the total electrolyte fluid. In some variations, the compound of Formula (I) is at least 1.5 wt % of the total electrolyte fluid. In some variations, the compound of Formula (I) is at least 2.0 wt % of the total electrolyte fluid. In some variations, the compound of Formula (I) is at least 2.5 wt % of the total electrolyte fluid. In some variations, the compound of Formula (I) is at least 3.0 wt % of the total electrolyte fluid. In some variations, the compound of Formula (I) is at least 3.5 wt % of the total electrolyte fluid. In some variations, the compound of Formula (I) is at least 4.0 wt % of the total electrolyte fluid. In some variations, the compound of Formula (I) is at least 4.5 wt % of the total electrolyte fluid. In some variations, the compound of Formula (I) is at least 5.0 wt % of the total electrolyte fluid. In some variations, the compound of Formula (I) is at least 5.5 wt % of the total electrolyte fluid. In some variations, the compound of Formula (I) is at least 6.0 wt % of the total electrolyte fluid. In some variations, the compound of Formula (I) is at least 6.5 wt % of the total electrolyte fluid. In some variations, the compound of Formula (I) is at least 7.0 wt % of the total electrolyte fluid. In some variations, the compound of Formula (I) is at least 7.5 wt % of the total electrolyte fluid. In some variations, the compound of Formula (I) is at least 8.0 wt % of the total electrolyte fluid. In some variations, the compound of Formula (I) is at least 8.5 wt % of the total electrolyte fluid. In some variations, the compound of Formula (I) is at least 9.0 wt % of the total electrolyte fluid. In some variations, the compound of Formula (I) is at least 9.5 wt % of the total electrolyte fluid.

In some variations, the compound of Formula (I) is less than or equal to 10.0 wt % of the total electrolyte fluid. In some variations, the compound of Formula (I) is less than or equal to 9.5 wt % of the total electrolyte fluid. In some variations, the compound of Formula (I) is less than or equal to 9.0 wt % of the total electrolyte fluid. In some variations, the compound of Formula (I) is less than or equal to 8.5 wt % of the total electrolyte fluid. In some variations, the compound of Formula (I) is less than or equal to 8.0 wt % of the total electrolyte fluid. In some variations, the compound of Formula (I) is less than or equal to 7.5 wt % of the total electrolyte fluid. In some variations, the compound of Formula (I) is less than or equal to 7.0 wt % of the total electrolyte fluid. In some variations, the compound of Formula (I) is less than or equal to 6.5 wt % of the total electrolyte fluid. In some variations, the compound of Formula (I) is less than or equal to 6.0 wt % of the total electrolyte fluid. In some variations, the compound of Formula (I) is less than or equal to 5.5 wt % of the total electrolyte fluid. In some variations, the compound of Formula (I) is less than or equal to 5.0 wt % of the total electrolyte fluid. In some variations, the compound of Formula (I) is less than or equal to 4.5 wt % of the total electrolyte fluid. In some variations, the compound of Formula (I) is less than or equal to 4.0 wt % of the total electrolyte fluid. In some variations, the compound of Formula (I) is less than or equal to 3.5 wt % of the total electrolyte fluid. In some variations, the compound of Formula (I) is less than or equal to 3.0 wt % of the total electrolyte fluid. In some variations, the compound of Formula (I) is less than or equal to 2.5 wt % of the total electrolyte fluid. In some variations, the compound of Formula (I) is less than or equal to 2.0 wt % of the total electrolyte fluid. In some variations, the compound of Formula (I) is less than or equal to 1.5 wt % of the total electrolyte fluid. In some variations, the compound of Formula (I) is less than or equal to 1.0 wt % of the total electrolyte fluid. In some variations, the compound of Formula (I) is less than or equal to 0.8 wt % of the total electrolyte fluid. In some variations, the compound of Formula (I) is less than or equal to 0.6 wt % of the total electrolyte fluid. In some variations, the compound of Formula (I) is less than or equal to 0.4 wt % of the total electrolyte fluid. In some variations, the compound of Formula (I) is less than or equal to 0.2 wt % of the total electrolyte fluid.

The electrolyte fluid includes an electrolyte solvent. The electrolyte solvent may be any type of electrolyte solvent suitable for battery cells. Non-limiting examples of the electrolyte solvents include propylene carbonate (PC), ethylene carbonate (EC), dimethyl carbonate (DMC), diethyl carbonate (DEC), ethyl-methyl carbonate (EMC), ethyl propionate (EP), butyl butyrate (BB), methyl acetate (MA), methyl butyrate (MB), methyl propionate (MP), propylene carbonate (PC), ethyl acetate (EA), propyl propionate (PP), butyl propionate (BP), propyl acetate (PA), and butyl acetate (BA), or combinations thereof.

The electrolyte fluid also has one or more electrolyte salts dissolved therein. The salt may be any type of salt suitable for battery cells. For example, and without limitation, salts for a lithium-ion battery cell include LiPF₆, LiBF₄, LiClO₄, LiN(SO₂F)₂, LiSO₃CF₃, LiN(SO₂CF₃)₂, LiBC₄O₈, Li[PF₃(C₂CF₅)₃], LiC(SO₂CF₃)₃, and any combinations thereof. Other salts are possible, including combinations of salts.

In some variations, the salt is at least 0.1 M in the total electrolyte fluid. In some variations, the salt is at least 0.2 M in the total electrolyte fluid. In some variations, the salt is at least 0.3 M in the total electrolyte fluid. In some variations, the salt is at least 0.4 M in the total electrolyte fluid. In some variations, the salt is at least 0.5 M in the total electrolyte fluid. In some variations, the salt is at least 0.6 M in the total electrolyte fluid. In some variations, the salt is at least 0.7 M in the total electrolyte fluid. In some variations, the salt is at least 0.8 M in the total electrolyte fluid. In some variations, the salt is at least 0.9 M in the total electrolyte fluid. In some variations, the salt is at least 1.0 M in the total electrolyte fluid. In some variations, the salt is at least 1.3 M in the total electrolyte fluid. In some variations, the salt is at least 1.6 M in the total electrolyte fluid. In some variations, the salt is at least 1.9 M in the total electrolyte fluid.

In some variations, the salt is less than or equal to 2.0 M in the electrolyte fluid. In some variations, the salt is less than or equal to 1.9 M in the electrolyte fluid. In some variations, the salt is less than or equal to 1.6 M in the electrolyte fluid. In some variations, the salt is less than or equal to 1.3 M in the electrolyte fluid. In some variations, the salt is less than or equal to 1.1 M in the electrolyte fluid. In some variations, the salt is less than or equal to 1.0 M in the electrolyte fluid. In some variations, the salt is less than or equal to 0.9 M in the electrolyte fluid. In some variations, the salt is less than or equal to 0.8 M in the electrolyte fluid. In some variations, the salt is less than or equal to 0.7 M in the electrolyte fluid. In some variations, the salt is less than or equal to 0.6 M in the electrolyte fluid. In some variations, the salt is less than or equal to 0.5 M in the electrolyte fluid. In some variations, the salt is less than or equal to 0.4 M in the electrolyte fluid. In some variations, the salt is less than or equal to 0.3 M in the electrolyte fluid. In some variations, the salt is less than or equal to 0.2 M in the electrolyte fluid.

In some variations, the electrolyte fluid can include one or more additives. In various aspects, the additives can include lithium difluoro(oxalato)borate (LiDFOB), pro-1-ene-1, 3-sultone (PES), methylene methanedisulfonate (MMDS), vinyl ethylene carbonate (VEC), propane sultone (PS), fluoroethylene carbonate (FEC), succinonitrile (SN), vinyl carbonate (VC), adiponitrile (ADN), ethyleneglycol bis(2-cyanoethyl)ether (EGPN), and/or 1,3,6-hexanetricarbonitrile (HTCN), in any combination, and in ranges of quantities.

In some variations, LiDFOB is at least 0.1 wt % of the total electrolyte fluid. In some variations, LiDFOB is at least 0.2 wt % of the total electrolyte fluid. In some variations, LiDFOB is at least 0.3 wt % of the total electrolyte fluid. In some variations, LiDFOB is at least 0.4 wt % of the total electrolyte fluid. In some variations, LiDFOB is at least 0.5 wt % of the total electrolyte fluid. In some variations, LiDFOB is at least 0.6 wt % of the total electrolyte fluid. In some variations, LiDFOB is at least 0.7 wt % of the total electrolyte fluid. In some variations, LiDFOB is at least 0.8 wt % of the total electrolyte fluid. In some variations, LiDFOB is at least 0.9 wt % of the total electrolyte fluid. In some variations, LiDFOB is at least 1.0 wt % of the total electrolyte fluid. In some variations, LiDFOB is at least 1.3 wt % of the total electrolyte fluid. In some variations, LiDFOB is at least 1.6 wt % of the total electrolyte fluid. In some variations, LiDFOB is at least 1.9 wt % of the total electrolyte fluid.

In some variations, LiDFOB is less than or equal to 2.0 wt % of the total electrolyte fluid. In some variations, LiDFOB is less than or equal to 1.9 wt % of the total electrolyte fluid. In some variations, LiDFOB is less than or equal to 1.3 wt % of the total electrolyte fluid. In some variations, LiDFOB is less than or equal to 1.3 wt % of the total electrolyte fluid. In some variations, LiDFOB is less than or equal to 1.1 wt % of the total electrolyte fluid. In some variations, LiDFOB is less than or equal to 1.0 wt % of the total electrolyte fluid. In some variations, LiDFOB is less than or equal to 0.9 wt % of the total electrolyte fluid. In some variations, LiDFOB is less than or equal to 0.8 wt % of the total electrolyte fluid. In some variations, LiDFOB is less than or equal to 0.7 wt % of the total electrolyte fluid. In some variations, LiDFOB is less than or equal to 0.6 wt % of the total electrolyte fluid. In some variations, LiDFOB is less than or equal to 0.5 wt % of the total electrolyte fluid. In some variations, LiDFOB is less than or equal to 0.4 wt % of the total electrolyte fluid. In some variations, LiDFOB is less than or equal to 0.3 wt % of the total electrolyte fluid. In some variations, LiDFOB is less than or equal to 0.2 wt % of the total electrolyte fluid.

In some variations, the amount of PES is at least 0.5 wt % of the total electrolyte fluid. In some variations, the amount of PES is at least 0.6 wt % of the total electrolyte fluid. In some variations, the amount of PES is at least 0.9 wt % of the total electrolyte fluid. In some variations, the amount of PES is at least 1.3 wt % of the total electrolyte fluid. In some variations, the amount of PES is at least 1.6 wt % of the total electrolyte fluid. In some variations, the amount of PES is at least 1.9 wt % of the total electrolyte fluid. In some variations, the amount of PES is at least 2.2 wt % of the total electrolyte fluid. In some variations, the amount of PES is at least 2.5 wt % of the total electrolyte fluid. In some variations, the amount of PES is at least 2.8 wt % of the total electrolyte fluid. In some variations, the amount of PES is at least 3.1 wt % of the total electrolyte fluid.

In some variations, the amount of PES is less than or equal to 3.5 wt % of the total electrolyte fluid. In some variations, the amount of PES is less than or equal to 3.1 wt % of the total electrolyte fluid. In some variations, the amount of PES is less than or equal to 2.8 wt % of the total electrolyte fluid. In some variations, the amount of PES is less than or equal to 2.5 wt % of the total electrolyte fluid. In some variations, the amount of PES is less than or equal to 2.2 wt % of the total electrolyte fluid. In some variations, the amount of PES is less than or equal to 1.9 wt % of the total electrolyte fluid. In some variations, the amount of PES is less than or equal to 1.6 wt % of the total electrolyte fluid. In some variations, the amount of PES is less than or equal to 1.3 wt % of the total electrolyte fluid. In some variations, the amount of PES is less than or equal to 1.1 wt % of the total electrolyte fluid. In some variations, the amount of PES is less than or equal to 0.9 wt % of the total electrolyte fluid. In some variations, the amount of PES is less than or equal to 0.6 wt % of the total electrolyte fluid.

In some variations, the amount of MMDS is at least 0.1 wt % of the total electrolyte fluid. In some variations, the amount of MMDS is at least 0.2 wt % of the total electrolyte fluid. In some variations, the amount of MMDS is at least 0.3 wt % of the total electrolyte fluid. In some variations, the amount of MMDS is at least 0.4 wt % of the total electrolyte fluid. In some variations, the amount of MMDS is at least 0.5 wt % of the total electrolyte fluid. In some variations, the amount of MMDS is at least 0.6 wt % of the total electrolyte fluid. In some variations, the amount of MMDS is at least 0.7 wt % of the total electrolyte fluid. In some variations, the amount of MMDS is at least 0.8 wt % of the total electrolyte fluid. In some variations, the amount of MMDS is at least 0.9 wt % of the total electrolyte fluid. In some variations, the amount of MMDS is at least 1.0 wt % of the total electrolyte fluid. In some variations, the amount of MMDS is at least 1.1 wt % of the total electrolyte fluid. In some variations, the amount of MMDS is at least 1.2 wt % of the total electrolyte fluid. In some variations, the amount of MMDS is at least 1.3 wt % of the total electrolyte fluid. In some variations, the amount of MMDS is at least 1.4 wt % of the total electrolyte fluid.

In some variations, the amount of MMDS is less than or equal to 1.5 wt % of the total electrolyte fluid. In some variations, the amount of MMDS is less than or equal to 1.4 wt % of the total electrolyte fluid. In some variations, the amount of MMDS is less than or equal to 1.3 wt % of the total electrolyte fluid. In some variations, the amount of MMDS is less than or equal to 1.2 wt % of the total electrolyte fluid. In some variations, the amount of MMDS is less than or equal to 1.1 wt % of the total electrolyte fluid. In some variations, the amount of MMDS is less than or equal to 1.0 wt % of the total electrolyte fluid. In some variations, the amount of MMDS is less than or equal to 0.9 wt % of the total electrolyte fluid. In some variations, the amount of MMDS is less than or equal to 0.8 wt % of the total electrolyte fluid. In some variations, the amount of MMDS is less than or equal to 0.7 wt % of the total electrolyte fluid. In some variations, the amount of MMDS is less than or equal to 0.6 wt % of the total electrolyte fluid. In some variations, the amount of MMDS is less than or equal to 0.5 wt % of the total electrolyte fluid. In some variations, the amount of MMDS is less than or equal to 0.4 wt % of the total electrolyte fluid. In some variations, the amount of MMDS is less than or equal to 0.3 wt % of the total electrolyte fluid. In some variations, the amount of MMDS is less than or equal to 0.2 wt % of the total electrolyte fluid.

In some variations, the amount of VEC is at least 0.1 wt % of the total electrolyte fluid. In some variations, the amount of VEC is at least 0.2 wt % of the total electrolyte fluid. In some variations, the amount of VEC is at least 0.3 wt % of the total electrolyte fluid. In some variations, the amount of VEC is at least 0.4 wt % of the total electrolyte fluid. In some variations, the amount of VEC is at least 0.5 wt % of the total electrolyte fluid. In some variations, the amount of VEC is at least 0.6 wt % of the total electrolyte fluid. In some variations, the amount of VEC is at least 0.7 wt % of the total electrolyte fluid. In some variations, the amount of VEC is at least 0.8 wt % of the total electrolyte fluid. In some variations, the amount of VEC is at least 0.9 wt % of the total electrolyte fluid.

In some variations, the amount of VEC is less than or equal to 0.9 wt % of the total electrolyte fluid. In some variations, the amount of VEC is less than or equal to 0.8 wt % of the total electrolyte fluid. In some variations, the amount of VEC is less than or equal to 0.7 wt % of the total electrolyte fluid. In some variations, the amount of VEC is less than or equal to 0.6 wt % of the total electrolyte fluid. In some variations, the amount of VEC is less than or equal to 0.5 wt % of the total electrolyte fluid. In some variations, the amount of VEC is less than or equal to 0.4 wt % of the total electrolyte fluid. In some variations, the amount of VEC is less than or equal to 0.3 wt % of the total electrolyte fluid. In some variations, the amount of VEC is less than or equal to 0.2 wt % of the total electrolyte fluid.

In some variations, the amount of FEC is at least 2 wt % of the total electrolyte fluid. In some variations, the amount of FEC is at least 4 wt % of the total electrolyte fluid. In some variations, the amount of FEC is at least 6 wt % of the total electrolyte fluid. In some variations, the amount of FEC is at least 8 wt % of the total electrolyte fluid. In some variations, the amount of FEC is less than or equal to 10 wt % of the total electrolyte fluid. In some variations, the amount of FEC is less than or equal to 8 wt % of the total electrolyte fluid. In some variations, the amount of FEC is less than or equal to 6 wt % of the total electrolyte fluid. In some variations, the amount of FEC is less than or equal to 4 wt % of the total electrolyte fluid.

In some variations, the amount of PS is at least 0.5 wt % of the total electrolyte fluid. In some variations, the amount of PS is at least 1.0 wt % of the total electrolyte fluid. In some variations, the amount of PS is at least 1.5 wt % of the total electrolyte fluid. In some variations, the amount of PS is at least 2.0 wt % of the total electrolyte fluid. In some variations, the amount of PS is at least 2.5 wt % of the total electrolyte fluid. In some variations, the amount of PS is at least 3.0 wt % of the total electrolyte fluid. In some variations, the amount of PS is at least 3.5 wt % of the total electrolyte fluid. In some variations, the amount of PS is at least 4.0 wt % of the total electrolyte fluid. In some variations, the amount of PS is at least 4.5 wt % of the total electrolyte fluid. In some variations, the amount of PS is at least 5.0 wt % of the total electrolyte fluid.

In some variations, the amount of PS is less than or equal to 6.0 wt % of the total electrolyte fluid. In some variations, the amount of PS is less than or equal to 5.5 wt % of the total electrolyte fluid. In some variations, the amount of PS is less than or equal to 5.0 wt % of the total electrolyte fluid. In some variations, the amount of PS is less than or equal to 4.5 wt % of the total electrolyte fluid. In some variations, the amount of PS is less than or equal to 4.0 wt % of the total electrolyte fluid. In some variations, the amount of PS is less than or equal to 3.5 wt % of the total electrolyte fluid. In some variations, the amount of PS is less than or equal to 3.0 wt % of the total electrolyte fluid. In some variations, the amount of PS is less than or equal to 2.5 wt % of the total electrolyte fluid. In some variations, the amount of PS is less than or equal to 2.0 wt % of the total electrolyte fluid. In some variations, the amount of PS is less than or equal to 1.5 wt % of the total electrolyte fluid. In some variations, the amount of PS is less than or equal to 1.0 wt % of the total electrolyte fluid.

In some variations, the amount of SN is at least 0.5 wt % of the total electrolyte fluid. In some variations, the amount of SN is at least 1.0 wt % of the total electrolyte fluid. In some variations, the amount of SN is at least 1.5 wt % of the total electrolyte fluid. In some variations, the amount of SN is at least 2.0 wt % of the total electrolyte fluid. In some variations, the amount of SN is at least 2.5 wt % of the total electrolyte fluid. In some variations, the amount of SN is at least 3.0 wt % of the total electrolyte fluid. In some variations, the amount of SN is at least 3.5 wt % of the total electrolyte fluid. In some variations, the amount of SN is at least 4.0 wt % of the total electrolyte fluid. In some variations, the amount of SN is at least 4.5 wt % of the total electrolyte fluid. In some variations, the amount of SN is at least 5.0 wt % of the total electrolyte fluid.

In some variations, the amount of SN is less than or equal to 6.0 wt % of the total electrolyte fluid. In some variations, the amount of SN is less than or equal to 5.5 wt % of the total electrolyte fluid. In some variations, the amount of SN is less than or equal to 5.0 wt % of the total electrolyte fluid. In some variations, the amount of SN is less than or equal to 4.5 wt % of the total electrolyte fluid. In some variations, the amount of SN is less than or equal to 4.0 wt % of the total electrolyte fluid. In some variations, the amount of SN is less than or equal to 3.5 wt % of the total electrolyte fluid. In some variations, the amount of SN is less than or equal to 3.0 wt % of the total electrolyte fluid. In some variations, the amount of SN is less than or equal to 2.5 wt % of the total electrolyte fluid. In some variations, the amount of SN is less than or equal to 2.0 wt % of the total electrolyte fluid. In some variations, the amount of SN is less than or equal to 1.5 wt % of the total electrolyte fluid. In some variations, the amount of SN is less than or equal to 1.0 wt % of the total electrolyte fluid.

In some variations, the amount of HTCN is at least 0.5 wt % of the total electrolyte fluid. In some variations, the amount of HTCN is at least 1.0 wt % of the total electrolyte fluid. In some variations, the amount of HTCN is at least 1.5 wt % of the total electrolyte fluid. In some variations, the amount of HTCN is at least 2.0 wt % of the total electrolyte fluid. In some variations, the amount of HTCN is at least 2.5 wt % of the total electrolyte fluid. In some variations, the amount of HTCN is at least 3.0 wt % of the total electrolyte fluid. In some variations, the amount of HTCN is at least 3.5 wt % of the total electrolyte fluid. In some variations, the amount of HTCN is at least 4.0 wt % of the total electrolyte fluid. In some variations, the amount of HTCN is at least 4.5 wt % of the total electrolyte fluid. In some variations, the amount of HTCN is at least 5.0 wt % of the total electrolyte fluid.

In some variations, the amount of HTCN is less than or equal to 6.0 wt % of the total electrolyte fluid. In some variations, the amount of HTCN is less than or equal to 5.5 wt % of the total electrolyte fluid. In some variations, the amount of HTCN is less than or equal to 5.0 wt % of the total electrolyte fluid. In some variations, the amount of HTCN is less than or equal to 4.5 wt % of the total electrolyte fluid. In some variations, the amount of HTCN is less than or equal to 4.0 wt % of the total electrolyte fluid. In some variations, the amount of HTCN is less than or equal to 3.5 wt % of the total electrolyte fluid. In some variations, the amount of HTCN is less than or equal to 3.0 wt % of the total electrolyte fluid. In some variations, the amount of HTCN is less than or equal to 2.5 wt % of the total electrolyte fluid. In some variations, the amount of HTCN is less than or equal to 2.0 wt % of the total electrolyte fluid. In some variations, the amount of HTCN is less than or equal to 1.5 wt % of the total electrolyte fluid. In some variations, the amount of HTCN is less than or equal to 1.0 wt % of the total electrolyte fluid.

The electrolyte solvent may also have a salt dissolved therein. The salt may be any type of salt suitable for battery cells. For example, and without limitation, salts for a lithium-ion battery cell include LiPF₆, LiBF₄, LiClO₄, LiSO₃CF₃, LiN(SO₂CF₃)₂, LiBC₄O₈, Li[PF₃(C₂CF₅)₃], and LiC(SO₂CF₃)₃. Other salts are possible, including combinations of salts.

In some variations, the salt is at least 0.1 wt % of the total electrolyte fluid. In some variations, the salt is at least 0.2 wt % of the total electrolyte fluid. In some variations, the salt is at least 0.3 wt % of the total electrolyte fluid. In some variations, the salt is at least 0.4 wt % of the total electrolyte fluid. In some variations, the salt is at least 0.5 wt % of the total electrolyte fluid. In some variations, the salt is at least 0.6 wt % of the total electrolyte fluid. In some variations, the salt is at least 0.7 wt % of the total electrolyte fluid. In some variations, the salt is at least 0.8 wt % of the total electrolyte fluid. In some variations, the salt is at least 0.9 wt % of the total electrolyte fluid. In some variations, the salt is at least 1.0 wt % of the total electrolyte fluid. In some variations, the salt is at least 1.3 wt % of the total electrolyte fluid. In some variations, the salt is at least 1.6 wt % of the total electrolyte fluid. In some variations, the salt is at least 1.9 wt % of the total electrolyte fluid.

### EXAMPLES

The Examples are provided for illustration purposes only. These examples are not intended to constrain any embodiment disclosed herein to any application or theory of operation.

Various battery cell properties were tested with the electrolyte fluid including Pyr14CNFSI, and compared to a battery cell with a control electrolyte fluid lacking Pyr14CNFSI. The composition of the control electrolyte fluid is show in Table 1.

TABLE 1

| Salt/M | Solvent wt % | | | | Additives wt % | | |
|---|---|---|---|---|---|---|---|
| LiPF6 | EC | PC | PP | EP | PS | FEC | SN |
| 1.2 | 20 | 10 | 45 | 25 | 4 | 7 | 3 |

### Example 1

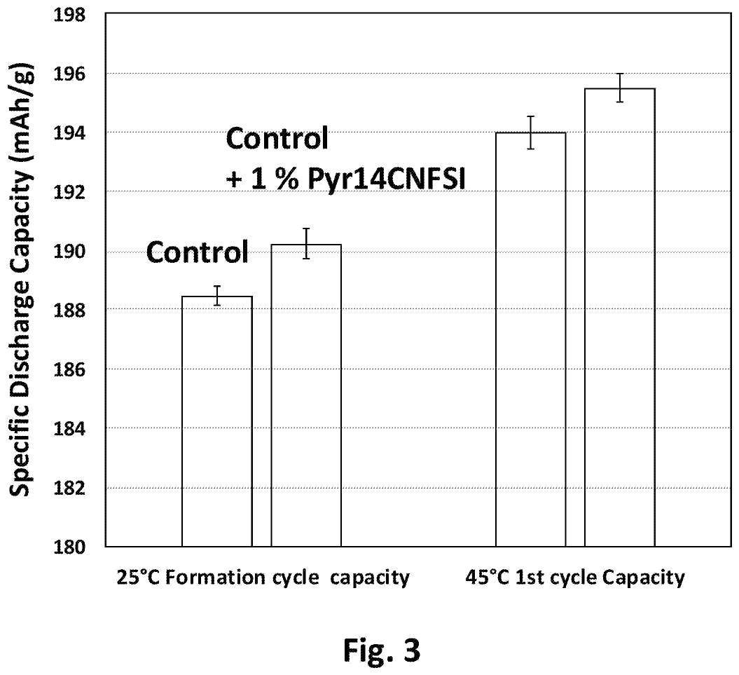

FIG. 3 depicts specific discharge capacity for a battery containing a control electrolyte formulation compared to a battery containing the control electrolyte formulation with a 1% Pyr14CNFSI. The specific discharge capacity of the formation cycle capacity at 25° C. and the first cycle capacity at 45° C. were both compared. In both cases, batteries containing electrolyte fluids having Pyr14CNFSI had a higher specific discharge capacity both at formation and after the first battery cycle.

### Example 2

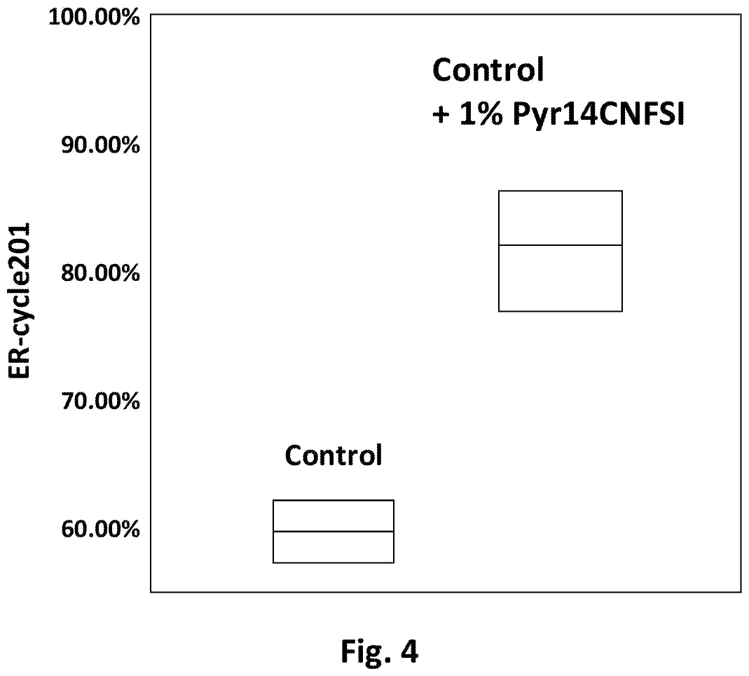

FIG. 4 depicts the cycling energy retention at cycle 200 for a battery operating at 45° C. of the control electrolyte as compared to the control including 1% Pyr14CNFSI. When the control electrolyte was used in the absence of Pyr14CNFSI, the energy retention at cycle 200 was approximately 60%. Upon the addition of 1% Pyr14CNFSI, the energy retention at cycle 200 was over 80%. The addition of Pyr14CNFSI resulted in a substantial increase in energy retention at high cycle times.

### Example 3

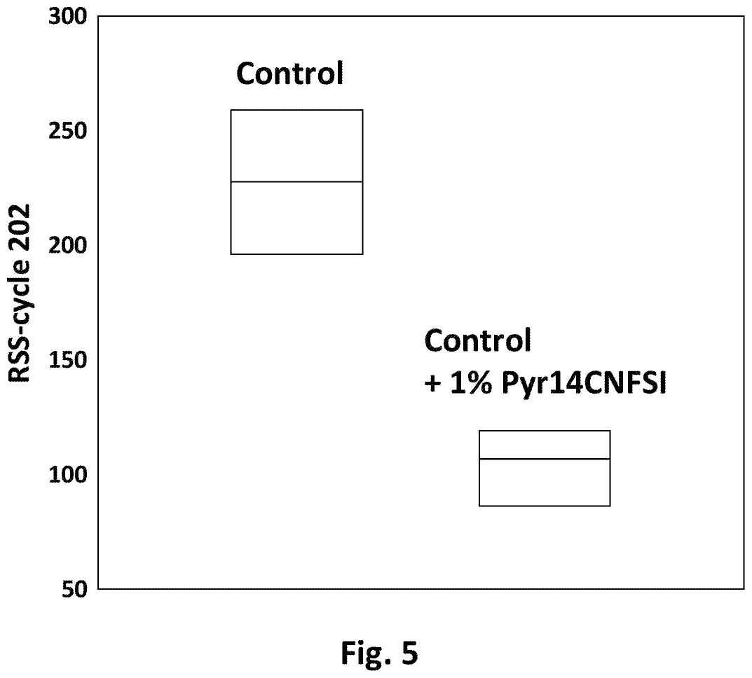

FIG. 5 shows the cycling RSS at cycle 202 at cycle 200 for a battery operating at 45° C. a battery having control electrolyte and the control electrolyte including 1% Pyr14CNFSI. The internal battery resistance was substantially higher additional cycles in the absence of the Pyr14CNFSI. In the absence of 1% Pyr14CNFSI, the resistance at cycle number 202 was from 200-250 When the electrolyte formulation includes 1% Pyr14CNFSI, the RSS was approximately 100 (ranging from 90 to 120).

### Example 4

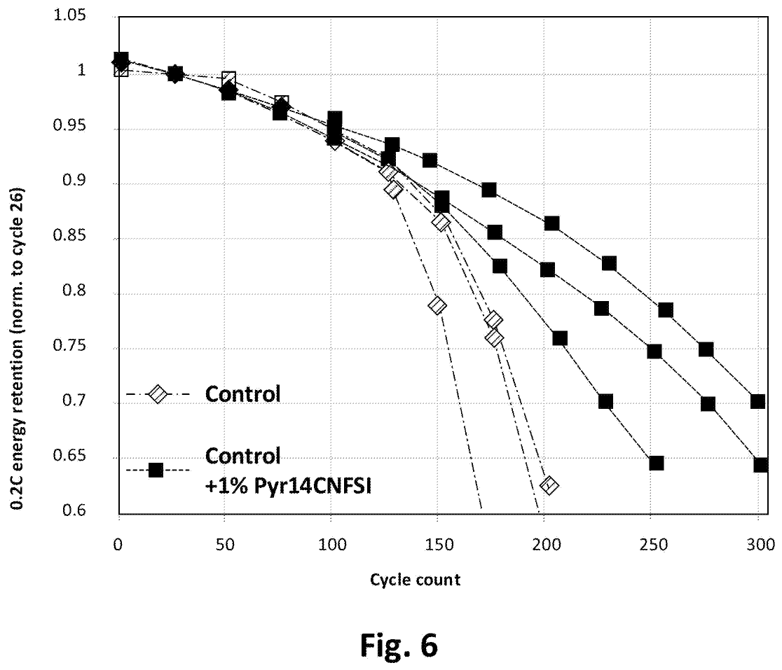

FIG. 6 is a plot of 0.2 C energy retention normalized to cycle 26 as a function of cycle count at 45° C. for a battery having a control electrolyte and an electrolyte including 1% Pyr14CNFSI. Three trials were measured with and without Pyr14CNFSI. The energy retention with and without Pyr14CNFSI was roughly similar through 75 cycles. However, the energy retention begins to fall precipitously in the absence of Pyr14CNFSI. The data demonstrate that the presence of Pyr14CNFSI in electrolytes substantially improves energy retention in battery cells as cycle count increases.

### Example 5

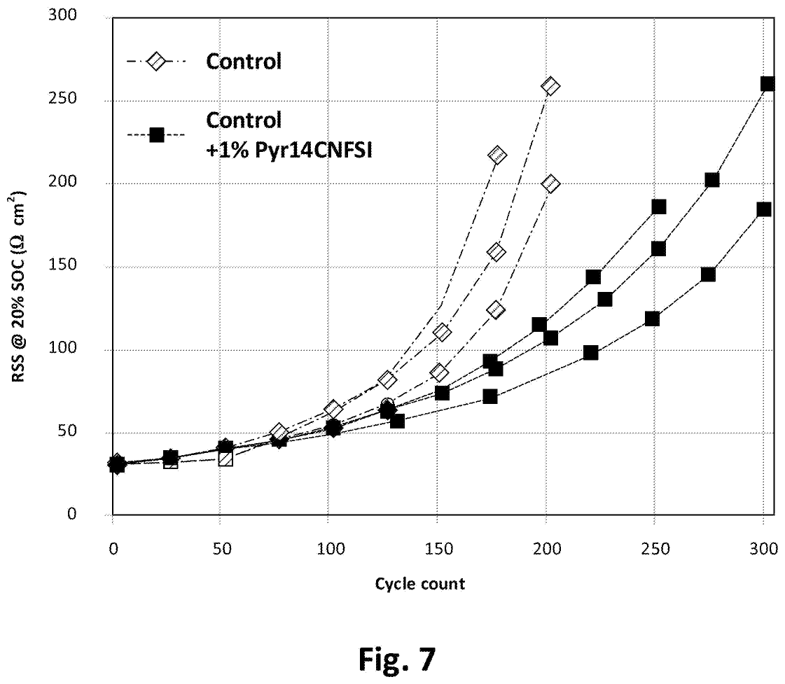

FIG. 7 depicts a plot of RSS as a function of battery cycle count at 45° C. for a battery having a control electrolyte and an electrolyte including 1% Pyr14CNFSI. Three trials were measured with and without Pyr14CNFSI. The RSS of the battery cells with and without Pyr14CNFSI was roughly similar through 75 cycles. However, the energy retention began to increase substantially in the absence of Pyr14CNFSI. The presence of Pyr14CNFSI in electrolyte formulations substantially lowered RSS in battery cells as cycle count increased.

The recovery capacity after storage was measured for battery cells having a control electrolyte fluid, the control electrolyte fluid with 1 wt % Pyr14FSI, and the control electrolyte fluid 1 wt % Pyr14CNFSI. As depicted in Table 2, batteries having a control electrolyte fluid and a control electrolyte fluid with Pyr14FSI had a lower recovery capacity than the recovery capacity of a battery in which the electrolyte fluid included Pyr14CNFSI.

TABLE 2

| | Remaining | Recovering Cap % | |
|---|---|---|---|
| Electrolyte ID | Cap % | Cycle 1 | Cycle 3 |
| 1 | 76.4 +/− 4.7 | 77.2 +/− 1.4 | 85.8 +/− 2.5 |
| El. 1 + 1% Pyr13FSI | 74.6 +/− 2.9 | 78.7 +/− 3.6 | |
| El. 1 + 1% | 83.8 +/− 0.7 | 85.4 +/− 0.2 | 92.6 +/− 1.4 |
| Pyr14CNFSI | | | |

The electrolyte fluids described herein can be valuable in battery cells, including those used in electronic devices and consumer electronic products. An electronic device herein can refer to any electronic device known in the art. For example, the electronic device can be a telephone, such as a cell phone, and a land-line phone, or any communication device, such as a smart phone, including, for example an iPhone®, an electronic email sending/receiving device. The electronic device can also be an entertainment device, including a portable DVD player, conventional DVD player, Blue-Ray disk player, video game console, music player, such as a portable music player (e.g., iPod®), etc. The electronic device can be a part of a display, such as a digital display, a TV monitor, an electronic-book reader, a portable web-browser (e.g., iPad®), watch (e.g., AppleWatch), or a computer monitor. The electronic device can also be a part of a device that provides control, such as controlling the streaming of images, videos, sounds (e.g., Apple TV®), or it can be a remote control for an electronic device. Moreover, the electronic device can be a part of a computer or its accessories, such as the hard drive tower housing or casing, laptop housing, laptop keyboard, laptop track pad, desktop keyboard, mouse, and speaker. The anode cells, lithium-metal batteries, and battery packs can also be applied to a device such as a watch or a clock.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. An electrolyte fluid electrolyte fluid comprising a compound of Formula (I): 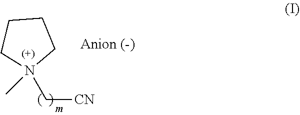
wherein m is an integer greater than or equal to 1 and less than or equal to 5.

2. The electrolyte fluid of claim 1, wherein m is greater than 2 and less than 4.

3. The electrolyte fluid of claim 1, wherein m is 3.

4. The electrolyte fluid of claim 1, wherein the anion is selected from N(SO₂F)₂, Cl⁻, AlCl₄⁻, PF₆⁻, BF₄⁻, NTf₂⁻, OTF, N(CN)₂—, HSO₄⁻, DCA⁻, CH₃COO⁻, CH₃CH₂OSO₃⁻, and CH₃SO₃⁻.

5. The electrolyte fluid of claim 1, wherein the compound is the compound of Formula (II):

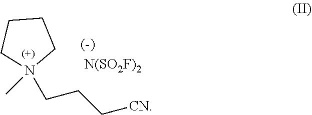

6. The electrolyte fluid of claim 1, wherein the compound is in an amount between 0.1 wt % and 10.0 wt % of the total electrolyte fluid.

7. The electrolyte fluid of claim 1, comprising an electrolyte salt selected from LiPF₆, LiBF₄, LiClO₄, LiSO₃CF₃, LiN(SO₂CF₃)₂, LiBC₄O₈, Li[PF₃(C₂CF₅)₃], LiC(SO₂CF₃)₃, and a combination thereof.

8. The electrolyte fluid of claim 7, wherein the salt comprises LiPF₆.

9. The electrolyte fluid of claim 7, wherein the salt is from 0.8 M to 1.6 M.

10. The electrolyte fluid of claim 1, comprising a solvent selected from ethylene carbonate (EC), dimethyl carbonate (DMC), diethyl carbonate (DEC), ethyl-methyl carbonate (EMC), ethyl propionate (EP), butyl butyrate (BB), methyl acetate (MA), ethyl acetate (EA), propyl propionate (PP), butyl propionate (BP), propyl acetate (PA), and butyl acetate (BA), and a combination thereof.

11. The electrolyte fluid of claim 10, wherein the solvent is selected from PC, EC, PP, EP, and a combination thereof.

12. The electrolyte fluid of claim 10, wherein the solvent comprises PC, EC, PP, and EP.

13. The electrolyte fluid of claim 10, wherein PC is from 2 to 20 wt % of the total electrolyte fluid, EC is from 5 to 40 wt % of the total electrolyte fluid, PP is from 20 to 70 wt % of the total electrolyte fluid, and/or EP is from 10 to 50 wt % of the total electrolyte fluid.

14. The electrolyte fluid of claim 1, comprising an additive selected from (LiDFOB), pro-1-ene-1, 3-sultone (PES), methylene methanedisulfonate (MMDS), propylene carbonate (PC), vinyl ethylene carbonate (VEC), propane sultone (PS), fluoroethylene carbonate (FEC), succinonitrile (SN), 1,3,6-hexanetricarbonitrile (HTCN), and a combination thereof.

15. The electrolyte fluid of claim 14, wherein the additive is selected from LidFOB, PES, MMDS, PS, FEC, SN, HTCN, and a combination thereof.

16. The electrolyte fluid of claim 15, wherein the additive comprises LidFOB, PES, MMDS, PS, FEC, SN, and HTCN.

17. A battery cell comprising:
a cathode comprising a cathode active material disposed on a cathode current collector;
an anode comprising an anode active material disposed on an anode current collector, the anode oriented towards the cathode such that the anode active material faces the cathode active material;
a separator disposed between the cathode active material and the anode active material; and
an electrolyte fluid according to claim 1 disposed between the cathode and anode.

18. The battery cell of claim 17, wherein the specific discharge capacity at 25° C. of the battery cell is higher than the specific discharge capacity at 25° C. of a battery cell comprising the electrolyte fluid that does not include a compound of Formula (I).

19. The battery cell of claim 17, wherein the energy retention of the battery cell at cycle 200 is at least 20% higher than the energy retention of a battery cell comprising the electrolyte fluid in the absence of the compound of Formula (I).

20. The battery cell of claim 17, wherein the RSS of the battery cell at cycle 200 is at least 40% decreased than the compared a battery cell comprising the electrolyte fluid in the absence of the compound of Formula (I).
